(19) United States
(12) Patent Application Publication
(10) Pub. No.: US 2026/0310973 A1
(43) Pub. Date: Oct. 8, 2026

(54) SYSTEMS AND METHODS FOR USING QSAR DATA TO ADAPT PRODUCTS FOR REGIONS

(71) Applicant: Mars, Incorporated, McLean, VA (US)

(72) Inventors: David W. STEFANY, McLean, VA (US); Mark NISBET, McLean, VA (US)

(21) Appl. No.: 19/480,565

(22) PCT Filed: May 10, 2024
(86) PCT No.: PCT/US2024/028899

§ 371 Date: Oct. 31, 2025

Related U.S. Application Data
(60) Provisional application No. 63/466,151, filed on May 12, 2023.

(51) Int. Cl. G06N 3/0895; G16C 60/00

(52) U.S. Cl. CPC G06N 3/0895; G16C 60/00

(57) ABSTRACT

A method for predicting a property of a product with substitute ingredients includes receiving data of an ingredient profile of a product, a molecular profile associated with the ingredient profile, and a formula profile for the product based on the ingredient profile; determining a set of molecular descriptors for the product based on the molecular profile; determining a set of material descriptors for the product based on the set of molecular descriptors; determining a set of formula descriptors for the product based on the set of material descriptors and the formula profile; generating a measured set of functional properties for the product using a machine-learning model based on the one or more formula descriptors; receiving one or more substitute ingredients for the product; and generating a predicted set of functional properties for the product through use of the machine-learning model based on the one or more substitute ingredients.

### CROSS-REFERENCE TO RELATED APPLICATION(S)

This application claims the benefit of priority to U.S. Provisional Application No. 63/466,151, filed on May 12, 2023, the entirety of which is incorporated herein by reference.

### TECHNICAL FIELD

Various embodiments of the present disclosure relate generally to systems and methods for using quantitative structure-activity relationship (QSAR) data to adapt products for regions and, more particularly, to systems and methods for predicting a property of a product with substitute ingredients using machine-learning based models based on a quantitative structure-activity relationship (QSAR) between the structure of substitute ingredients and their properties.

### BACKGROUND

Functional properties of food products are a result of the ingredients used and the process applied to create complex interactions between the ingredients. The ingredients used in the food products may impart the product with various functional properties such as texture, plasticity, softness, hardness, spreadability, satiety, and mouthfeel. Substitution of one or more ingredients of a product may change one or more functional properties of the product.

The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

In some aspects, the techniques described herein relate to a computer-implemented method for predicting a property of a product with substitute ingredients, the method including: receiving, by one or more processors, data associated with a product, the data including an ingredient profile of the product, a molecular profile associated with the ingredient profile, and a formula profile for the product based on the ingredient profile; determining, by the one or more processors, a set of molecular descriptors for the product based on the molecular profile; determining, by the one or more processors, a set of material descriptors for the product based on the set of molecular descriptors; determining, by the one or more processors, a set of formula descriptors for the product based on the set of material descriptors and the formula profile; and generating, by the one or more processors, a measured set of functional properties for the product through training of a machine-learning model based on the one or more formula descriptors; receiving, by the one or more processors, one or more substitute ingredients for the product to be evaluated with the machine-learning model; and generating, by the one or more processors, a predicted set of functional properties for the product through use of the machine-learning model based on the one or more substitute ingredients.

In some aspects, the techniques described herein relate to a computer-implemented method, further including: prior to generating the predicted set of functional properties and after receiving the one or more substitute ingredients, adjusting, by the one or more processors, the formula profile and the set of molecular descriptors based on the one or more substitute ingredients; adjusting, by the one or more processors, the set of material descriptors based on the adjusted set of molecular descriptors; and adjusting, by the one or more processors, the set of formula descriptors based on the adjusted set of material descriptors and the adjusted formula profile, wherein generating the predicted set of functional properties through use of the machine-learning model is further based on the adjusted set of formula descriptors.

In some aspects, the techniques described herein relate to a computer-implemented method, further including: generating a substitute ingredient profile including the one or more substitute ingredients for the product based on a similarity between the predicted set of functional properties and the measured set of functional properties, wherein the similarity is determined based on an evaluation of a distance metric between the predicted set of functional properties and the measured set of functional properties, the distance metric including one or more of: a euclidean distance metric or a cosine distance metric.

In some aspects, the techniques described herein relate to a computer-implemented method, wherein the one or more substitute ingredients include one or more of: a different weight composition of one or more like ingredients contained in the ingredient profile or one or more new ingredients not contained in the ingredient profile.

In some aspects, the techniques described herein relate to a computer-implemented method, wherein the product includes one of: a toffee candy, a chocolate, a nougat, or a caramel.

In some aspects, the techniques described herein relate to a computer-implemented method, wherein the machine-learning model includes a quantitative structure-activity relationship (QSAR) model, the QSAR model including one of: a linear regression model, a stepwise linear regression model, a partial least squares regression model, a principal components regression model, a support vector regression model, neural networks, a multivariate adaptive regression model, a multivariate linear regression model, or a random forest model.

In some aspects, the techniques described herein relate to a computer-implemented method, wherein the machine-learning model includes one of: a polynomial regression model, a generalized additive model, a Bayesian additive regression tree model (BART), a classification and regression tree model (CART), a multi-layer perceptron model (MLP), a recurrent neural network model (RNN), or a convolutional neural network model (CNN).

In some aspects, the techniques described herein relate to a computer-implemented method, wherein training the machine-learning model includes use of a train/test split, use of hyperparameters tuned using a multi-fold repeated cross validation, or use of a variable selection technique.

In some aspects, the techniques described herein relate to a computer-implemented method, wherein the multi-fold repeated cross validation ranges in value between 5 and 10 repeated cross validations, and wherein the variable selection technique includes one or more of: a recursive feature elimination, a backward or forward stepwise selection, simulated annealing, or a genetic algorithm search.

In some aspects, the techniques described herein relate to a computer-implemented method, wherein the ingredient profile includes ingredient data of individual ingredients used in manufacturing the product, the ingredient data including composition of the individual ingredients, the composition including one or more of: a carbohydrate profile, a fat profile, or a protein composition.

In some aspects, the techniques described herein relate to a computer-implemented method, wherein the set of molecular descriptors includes molecular data associated with individual ingredients contained in the ingredient profile, the molecular data including one or more of: composition properties of the individual ingredients, electronic properties of the individual ingredients, geometric properties of the individual ingredients, topological properties of the individual ingredients, or quantum mechanical properties of the individual ingredients.

In some aspects, the techniques described herein relate to a computer-implemented method, wherein the formula profile includes partial or full formulation data of the product based on the ingredient profile, the partial or full formulation data including a percentage composition of individual ingredients contained in the ingredient profile.

In some aspects, the techniques described herein relate to a computer-implemented method, wherein the measured set of functional properties and the predicted set of functional properties include organoleptic properties of the product.

Additional objects and advantages of the disclosed embodiments will be set forth in part in the description that follows, and in part will be apparent from the description, or may be learned by practice of the disclosed embodiments. The objects and advantages of the disclosed embodiments will be realized and attained by means of the elements and combinations particularly pointed out in the appended claims.

It is to be understood that both the foregoing general description and the following detailed description are exemplary and explanatory only and are not restrictive of the disclosed embodiments, as claimed.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 depicts an exemplary environment that may be utilized with techniques presented herein, according to one or more embodiments.

FIG. 2 depicts an exemplary flowchart of an exemplary quantitative structure-activity relationship (QSAR) modeling process for predicting a property of a product with substitute ingredients.

FIG. 3 depicts a flowchart illustrating an exemplary method for measuring a functional property of a product with a machine-learning model according to various embodiments.

FIG. 4 depicts a flowchart of an exemplary method for predicting a functional property of a product with substitute ingredients, using a trained machine-learning model discussed with respect to FIG. 3, according to various embodiments.

FIG. 5 illustrates an implementation of a computer system that may execute techniques presented herein according to various embodiments.

FIG. 6 is a diagram of training, deploying, and updating an artificial intelligence (AI) model.

FIG. 7 is a diagram of an example process for training the AI model.

FIG. 8 is a diagram of an example process for new formula performance prediction.

FIG. 9 is a diagram of an example process for generating information identifying a new ingredient for a product.

### DETAILED DESCRIPTION OF EMBODIMENTS

Various embodiments of the present disclosure relate generally to systems and methods for using quantitative structure-activity relationship (QSAR) data to adapt products for regions and, more particularly, to systems and methods for predicting a property of a product with substitute ingredients using machine-learning based models based on a quantitative structure-activity relationship (QSAR) between the structure of substitute ingredients and their properties.

The functional or sensory properties of food products (“products”) may be impacted by a result of the ingredients used for the product and the process applied to create complex interactions between the ingredients used. Products often need to be reformulated for a variety of reasons, to comply with regulations, improved nutrition properties, ingredient availability, or cost optimization. The difficulty in reformulating includes predicting how ingredient substitutions may change the functional properties of the product.

According to various embodiments, an artificial intelligence (AI) application may use quantitative structure-activity relationship (QSAR) data and other physical properties to predict what ingredients could be suitable replacements of a product to match organoleptic properties of the product based on using machine-learning models configured to measure performance of a substitute ingredient profile of the product based on a similarity between a predicted set of functional properties and a measured set of functional properties.

According to one example, the desire may be to make a reduced-sugar toffee candy to match the texture of a full-sugar version. Toffee is comprised of corn syrup, sugar, starch, palm kernel oil, colors and flavors. Based on the molecular weights and other physical properties related to the carbohydrates, a trained machine-learning model may suggest possible substitute ingredients for the reduced-sugar toffee candy to match the texture of the full-sugar version. For example, the ingredients for the full-sugar version may include corn syrup 26DE (% w/w, 20-50) sucrose (% w/w, 40-60), palm kernel oil (% w/w, 5-10), starch (% w/w, 0.1-0.5), and color/flavor (% w/w, 1-2). The trained machine-learning model may suggest substitute ingredients for replacement of the corn syrup 26DE (% w/w, 20-50) and the sucrose (% w/w, 40-60), such as corn syrup 42 (% w/w, 10-20), soluble fiber (% w/w, 10-30), sucrose (% w/w, 5-20), and allulose (% w/w, 10-30).

According to one example, the desire may be to implement fat replacement for a chocolate. A chocolate may include cocoa butter, cocoa solids, and sugar as the ingredients. To match texture or other functional properties of the chocolate, a trained machine-learning model may suggest substitute ingredients for the chocolate in replacement of the cocoa butter, such as shea butter and fractionated palm oil.

According to various embodiments, databases used for training the machine-learning model may include a molecule database that includes single small molecule data or macromolecule data along with calculated chemical predictors from the Chemistry Development Kit (CDK). The molecular descriptors include composition data, electronic data, geometric data, topological data, and quantum mechanical properties, among others.

According to various embodiments, the databases used may include an ingredient database that includes data relating to appropriate ingredients used in the manufacture of confectionery products along with the composition of one or more ingredients of a product. The composition may include a carbohydrate profile, a fat profile, or a protein composition.

According to various embodiments, the databases used may include a formula database that contains a full or partial formulation of a confectionery product or component (e.g., toffee, nougat, caramel), including ingredients and percentages thereof.

According to various embodiments, for training a machine-learning model, chemical predictors may be generated from molecular structures using an API to the CDK and then stored in the molecule database. The ingredient database may be joined to the molecule database descriptors and summarized to create a material descriptor. The descriptors may be joined with the formulation data and summarized to create a formula descriptor. A regression model may be fitted to predict the various functional properties from the formula descriptors. The performance of the machine-learning model may be measured using a common regression metric such as RMSE and R-squared to select the best model.

According to various embodiments, application of the product properties model may be run through an inference pipeline. A proposed formulation (using known ingredients) may be assessed as an input to the pipeline. The formula descriptors are generated from the stored material descriptor data and passed to the machine-learning model. The machine-learning model may predict the different performance measures of the product with the substitute or proposed formulation.

According to various embodiments, a different inference pipeline is proposed to evaluate novel ingredients. This approach includes creation of a mixture design to create new ingredients “in-silico” using a range of molecular combinations (e.g., novel starch derivatives). A mixture design is created to make a collection of ingredients and these are joined with the new chemical descriptor data to make a new set of material descriptors. A set of formulas specifying the incorporation of new ingredients is then joined with the expanded material descriptors to adjust the formula descriptors. These are passed to the machine-learning model, which can predict performance of the product based on the new or substitute ingredients. The output of the machine-learning model can be evaluated against a desired performance profile by a distance metric (e.g., Euclidean distance, cosine distance, etc.), and the closest matching formula may identify a potential novel ingredient substitution.

Examples of QSAR models include, but are not limited to regression (e.g., linear regression, stepwise linear regression, partial least squares regression, principal components regression, support vector regression, neural networks, multivariate adaptive regression, multivariate linear regression, random forests, etc.).

Other models for regression include polynomial regression, generalized additive models, Bayesian additive regression trees (BART), classification and regression trees (CART), neural network models including, but not limited to, multi-layer perceptron (MLP), recurrent neural networks (RNN), and convolutional neural networks (CNN).

The embodiments of the present disclosure are directed to solving, mitigating, or rectifying the above-mentioned issues by determining machine-learning models that are configured to predict and measure performance of a substitute ingredient profile of a product. The machine-learning models may use an initial set of data gathered at an initial point in time. The systems and methods of the present disclosure may be used to predict and measure substitute ingredient performance for a product, with specific regard to functional attributes or properties of the product, such as one or more organoleptic properties. The machine-learning models of the present disclosure may include regression models. In various implementations, any suitable regression model may be utilized, including but not limited to, linear, stepwise linear, partial least squares, principal components, support vector (SVR), neural networks, multivariate adaptive, multivariate linear, random forest, polynomial, generalized additive, Bayesian additive regression trees (BART), classification and regression tree (CART), or neural network models such as multi-layer perception (MLP), and recurrent neural networks (RNN), convolutional neural networks (CNN). Based on the prediction, a user may determine if a substitute ingredient profile or a formula for a product is suitable for implementation based on performance of one or more functional properties.

Although the models and embodiments described herein are directed to food products, the models of the present disclosure are applicable to a variety of edible and non-edible products.

The terminology used below may be interpreted in its broadest reasonable manner, even though it is being used in conjunction with a detailed description of certain specific examples of the present disclosure. Indeed, certain terms may even be emphasized below; however, any terminology intended to be interpreted in any restricted manner will be overtly and specifically defined as such in this Detailed Description section. Both the foregoing general description and the following detailed description are exemplary and explanatory only and are not restrictive of the features, as claimed.

In the detailed description herein, references to “embodiment,” “an embodiment,” “one non-limiting embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

In general, terminology can be understood at least in part from usage in context. For example, terms, such as “and”, “or”, or “and/or,” as used herein can include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, can be used to describe any feature, structure, or characteristic in a singular sense or can be used to describe combinations of features, structures or characteristics in a plural sense. Similarly, terms, such as “a,” “an,” or “the,” again, can be understood to convey a singular usage or to convey a plural usage, depending at least in part upon context. In addition, the term “based on” can be understood as not necessarily intended to convey an exclusive set of factors and can, instead, allow for existence of additional factors not necessarily expressly described, again, depending at least in part on context.

As used herein, the terms “comprises,” “comprising,” or any other variation thereof are intended to cover a non-exclusive inclusion, such that a process, method, composition, article, or apparatus that comprises a list of elements does not include only those elements, but may include other elements not expressly listed or inherent to such process, method, composition, article, or apparatus. The term “exemplary” is used in the sense of “example” rather than “ideal.” As used herein, the singular forms “a,” “an,” and “the” include plural reference unless the context dictates otherwise. Relative terms such as “about,” “substantially,” and “approximately” refer to being nearly the same as a referenced number or value, and should be understood to encompass a variation of ±5% of a specified amount or value.

As used herein, a “machine-learning model” generally encompasses instructions, data, and/or a model configured to receive input, and apply one or more of a weight, bias, classification, or analysis on the input to generate an output. The output may include, for example, a classification of the input, an analysis based on the input, a design, process, prediction, or recommendation associated with the input, or any other suitable type of output. A machine-learning model is generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine-learning model may operate on an input linearly, in parallel, via a network (e.g., a neural network), or via any suitable configuration.

The execution of the machine-learning model may include deployment of one or more machine learning techniques, such as linear regression, logistical regression, random forest, gradient boosted machine (GBM), deep learning, a deep neural network, etc. Supervised and/or unsupervised training may be employed. For example, supervised learning may include providing training data and labels corresponding to the training data, e.g., as ground truth. Unsupervised approaches may include clustering, classification or the like. Any suitable type of training may be used, e.g., stochastic, gradient boosted, random seeded, recursive, epoch or batch-based, etc.

Certain non-limiting embodiments are described below with reference to block diagrams and operational illustrations of methods, processes, devices, and apparatus. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

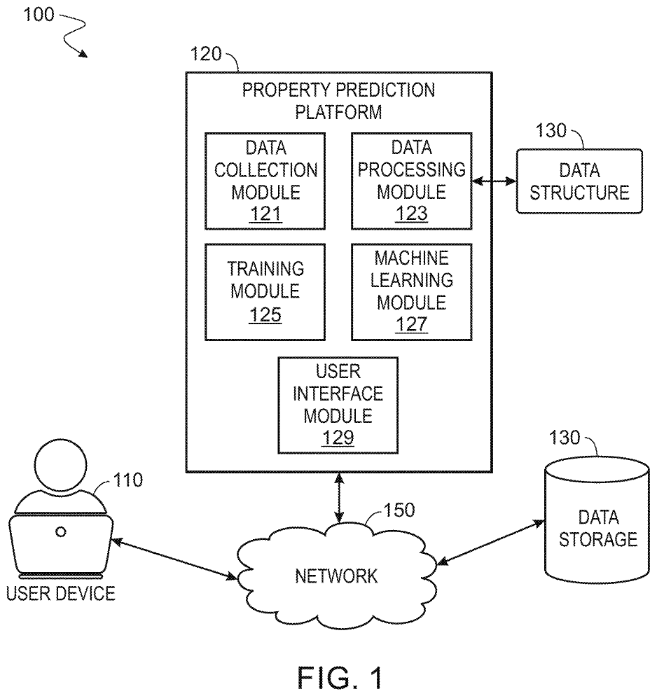

Referring now to the drawings, FIG. 1 depicts an exemplary environment 100 that may be utilized with the techniques presented herein. A user device 110, a property prediction platform 120, and a data storage 140 may communicate across a network 150. The user device 110 may be associated with a user, e.g., a user associated with one or more of generating, training, tuning or using a machine-learning model for predicting a property of a product with substitute ingredients. A user may use the user device 110 to interact with the user interface module 129 of the property prediction platform 120.

The property prediction platform 120 may be a platform with multiple interconnected components. The property prediction platform 120 may include one or more servers, intelligent networking devices, computing devices, components, and corresponding software for determining functional attributes, analyzing relationships, determining machine-learning models configured to predict functional properties based on one or more substitute ingredients implemented in a product.

The property prediction platform 120 comprises data collection module 121, data processing module 123, training module 125, machine learning model 127, and user interface module 129.

The data collection module 121 may receive an input from user device 110. In some examples, the input may include data associated with a product. In examples, the product may be an edible product, such as chocolate or toffee. The data associated with the product may include analytical data obtained from analytical testing of ingredients of the product or data about the product received from a database. The data received about the product may include an ingredient profile associated with the product, a molecular profile associated with the ingredient profile of the product, and a formula profile associated with the product. The ingredient profile data may include various individual ingredients contained in the product and may be received from an ingredient database.

The molecule profile may include molecular structures of individual ingredients contained in the ingredient profile. Molecular descriptors may be determined from the molecular profile, and may include properties and/or categories such as, composition, electronic, geometric, topological, and quantum mechanical properties, among others. In some cases, the molecular descriptors may be received from a molecule database comprised of single small molecule data or macromolecule data along with calculated chemical predictors from the chemistry development kit (CDK).

The formula profile may be associated with a formulation of the individual ingredients to make up the product. The formula profile may be received from a formula database that contains a full or partial formulation of a confectionery product or component including ingredients and percentages thereof.

In certain examples, a user may have the option to enter, via a user device 110, data associated with ingredients of a product for which a functional property is desired to be predicted and the data collection module 121 may receive the input. Additionally or alternatively, the data collection module 121 may receive (e.g., retrieve) the data associated with the ingredients of the product from the data storage 140.

Data processing module 123 may process data collected by data collection module 121. Processing the data collected may include determining associated molecular descriptors for the ingredients of the product. Once the molecular descriptors are determined or received, material descriptors for the product may be determined based on the molecular descriptors for the ingredient profile. For example, a material descriptor may be determined for each individual ingredient contained in the ingredient profile. Once the material descriptors are determined, formula descriptors for the product may be determined based on the material descriptors and the formula profile. In examples, the formula descriptors are input into a machine-learning model to train the machine-learning model to predict various functional properties of the product.

Once the machine-learning model is trained, one or more substitute ingredients for the product may be received to predict functional properties through the trained machine-learning model based on the one or more substitute ingredients. To predict the functional properties based on the substitute ingredients, the formula profile and the molecular descriptors are adjusted to include molecular and formulaic data associated with the one or more substitute ingredients. Next, the material descriptors are adjusted based on the adjusted set of molecular descriptors. Next, the formula descriptors are adjusted based on the adjusted material descriptors and the adjusted formula profile. Next, the trained machine-learning model may predict functional properties of the product with the substituted ingredients based on the adjusted formula descriptors.

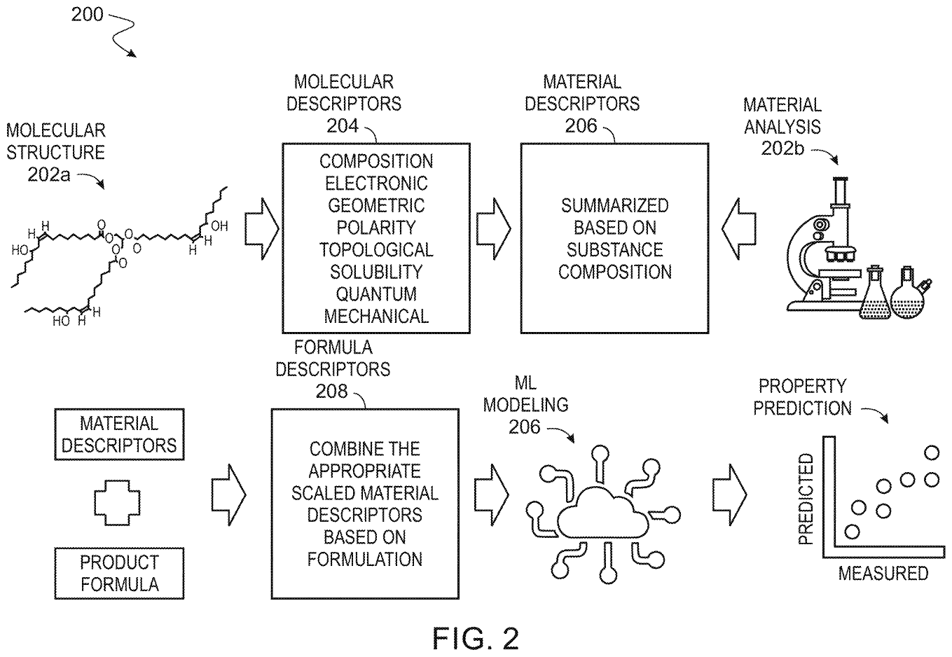

In some examples, the data processing module 123 may access data that is associated with a product from a database or another information source (e.g., data storage 140). Further details regarding computation of a predicted profile response are provided below in reference to FIG. 2.

In some embodiments, the data processing module 123 generates a data structure 130. Data structure 130 may include each ingredient of the product and the associated data, including an ingredient profile, molecular descriptors associated with the ingredient profile, a formula profile of the product based on the ingredient profile, material descriptors based on the molecular descriptors, and formula descriptors for the product based on the material descriptors and the formula profile. In at least one example, the data structure 130 may be a table.

Training module 125 may provide learning, or training to machine learning model 127 by providing training data, e.g., data from other modules that contains input (e.g., features) and correct output (e.g., labels), to allow machine learning model 127 to learn over time. For example, training module 125 may receive data from data structure 130 generated by the data processing module 123. The training may be performed based on the deviation of a processed result from a documented result when the inputs are fed into machine learning model 127, e.g., an algorithm measures its accuracy through the loss function, adjusting until the error has been sufficiently minimized. Training module 125 may conduct the training in any suitable manner, e.g., in batches, and may include any suitable training methodology. Training may be performed periodically, and/or continuously, e.g., in real-time or near real-time. Further details of training a machine learning model are provided below.

Machine learning model 127 may receive the training data from training module 125 to learn relationships between sensory attributes and shelf life progression. The ordering of the training data may be randomized during training. Machine learning model 127 may visualize the training data to identify relevant relationships between different variables and identify any data imbalances. The training data may be split into two parts where one part is for training the model and the other part is for validating the trained model, de-duplicating, normalizing, correcting errors in the training data, and so on. In some examples, machine learning model 127 may receive data directly from data structure 130. Machine learning model 127 may implement various machine learning techniques (e.g., random forest, k-nearest neighbor, partial least squares regression, principal component regression, etc.) discussed in the present disclosure.

User interface module 129 may enable a presentation of a graphical user interface (GUI) in user device 110. User interface module 129 may comprise a variety of interfaces, for example, interfaces for data input and output devices, referred to as I/O devices, storage devices, and the like.

Data storage 140 may store and manage data associated with the molecular descriptors, material descriptors, and the formula descriptors. Data storage 140 may store the data structure 130 generated by the data processing module 123. Data storage 140 may also store any information provided by a user via user device 110. In addition, data storage 140 may store data structure 130 generated by the property prediction platform 120. In some examples, data storage 140 may include a machine-learning based training database with pre-defined mapping defining a relationship between various input parameters and output parameters based on various statistical methods. The training database may include machine-learning algorithms to learn mappings between sensory attributes and shelf life progression. In some examples herein, the training database is routinely updated and/or supplemented based on machine learning methods.

FIG. 2 depicts an exemplary flowchart of an exemplary quantitative structure-activity relationship (QSAR) modeling process 200 for predicting a property of a product with substitute ingredients. The property prediction platform 120 may operate according to the QSAR modeling process 200.

A material analysis 202b may be conducted on an ingredient profile of a product. The ingredient profile may include one or more individual ingredients that make up the product. For example, the material analysis 202b may include first receiving a molecular structure 202a of the individual ingredients of the ingredient profile. In some examples, molecular structures 202a may be represented in a molecular structure file format, such as SMILES (the Simplified Molecular Input Line Entry System). In various examples, the molecular structures 202a may be a part of a molecular profile associated with the ingredient profile. Next, molecular descriptors 204 may be determined based on the molecular structures 202a, and may include 2D descriptors, 3D descriptors, or combinations thereof. Exemplary molecular descriptors 204, which may be determined may include: composition, electronic, geometric, polarity, topological, solubility, quantum, and mechanical. In some examples, a molecular descriptor may be based on a combination of molecular descriptors and may be classified as a hybrid descriptor. For example, a hybrid descriptor may be computed based on a combination of molecular descriptors from one or more of the categories described above.

After molecular descriptors 204 are determined for an ingredient profile, material descriptors 206 for the ingredient profile may be determined based on the molecular descriptors 204. For example, the molecular descriptors 204 may be scaled by multiplying the molecular descriptors 204 by a percentage composition of individual ingredients contained in the ingredient profile.

The material analysis 202b may also include analytical testing methods that are performed on each ingredient from the ingredient profile to measure and analyze a desired ingredient property. The analytical testing methods may include differential scanning calorimetry (DSC), nuclear magnetic resonance (NMR) spectroscopy, iodine value, oil stability index (OSI), and mettler drop point. Additional analytical testing methods include fatty acid profile analysis by GC or GC/MS, sugar profile analysis by HPLC or HPLC/MS, amino acid analysis, other hyphenated chromatographic methods (HPLC, UHPLC, GC combined with MS, UV, fluorescence, DAD, FID, MS-MS) to identify the composition of ingredients, and spectroscopic methods including NIR, FTIR, or Raman spectroscopy. The analytical testing methods may include analytical measurements that focus on texture (e.g., Farinograph, TA. TXPlus, or the like). The analytical testing methods may include methods to measure the rheological and viscosity properties of the materials using a rotational shear or extensional rheometer and viscometer, respectively. Different viscometers may be used for measurement of fluid viscosity including, but not limited to, rotational, vibrational, falling sphere, or U-tube viscometers.

Next, material descriptors 204 are analyzed together with a formula profile of a product to determine formula descriptors 208. The formula descriptors 208 include a combination of the scaled material descriptors 204 based on the formula profile.

A machine learning model 210 may be trained to generate a functional property of a product based on the formula descriptors 208. The trained machine learning model 210 may be configured to predict functional properties of the product with substitute ingredients. The predicted functional properties may be compared against the measured functional properties to determine a similarity. Based on the similarity, a substitute ingredient profile for the product can be generated, which includes the substitute ingredients.

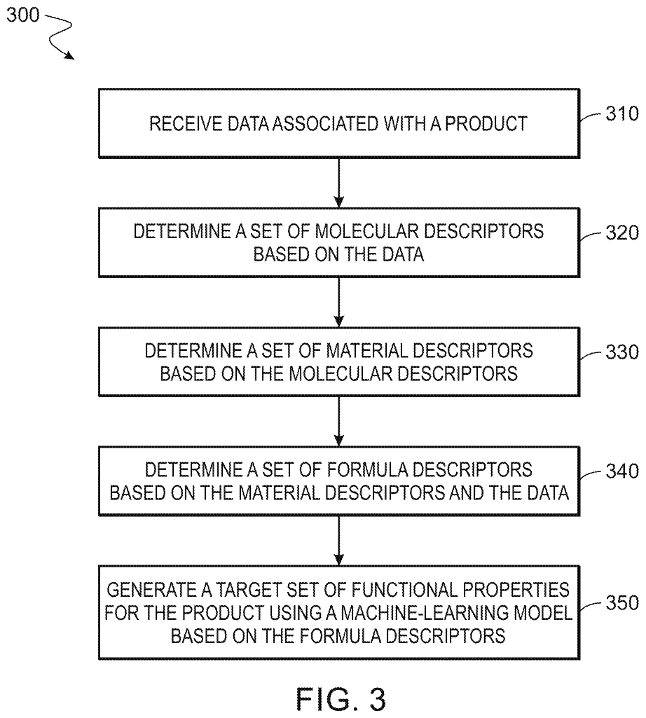

FIG. 3 depicts a flowchart illustrating an exemplary method for measuring a functional property of a product with a machine-learning model according to various embodiments. The method 300 may be performed by the property prediction platform 120 of FIG. 1.

In step 310, data associated with a product may be received. The data associated with the product may include analytical data obtained from analytical testing of ingredients of the product or data about the product received from a database. The data received about the product may include an ingredient profile associated with the product, a molecular profile associated with the ingredient profile of the product, and a formula profile associated with the product. The ingredient profile data may include various individual ingredients contained in the product and may be received from an ingredient database. The molecule profile may include molecular structures of individual ingredients contained in the ingredient profile.

A material analysis 202b (shown in FIG. 2) may be conducted on an ingredient profile of a product. The ingredient profile may include one or more individual ingredients that make up the product. For example, the material analysis 202b may include first receiving a molecular structure 202a of the individual ingredients of the ingredient profile. In some examples, molecular structures 202a may be represented in a molecular structure file format, such as SMILES (the Simplified Molecular Input Line Entry System). In various examples, the molecular structures 202a may be a part of a molecular profile associated with the ingredient profile.

The material analysis 202b may also include analytical testing methods that are performed on each ingredient from the ingredient profile to measure and analyze a desired ingredient property. The analytical testing methods may include differential scanning calorimetry (DSC), nuclear magnetic resonance (NMR) spectroscopy, iodine value, oil stability index (OSI), and mettler drop point. Additional analytical testing methods include fatty acid profile analysis by GC or GC/MS, sugar profile analysis by HPLC or HPLC/MS, amino acid analysis, other hyphenated chromatographic methods (HPLC, UHPLC, GC combined with MS, UV, fluorescence, DAD, FID, MS-MS) to identify the composition of ingredients, and spectroscopic methods including NIR, FTIR, or Raman spectroscopy. The analytical testing methods may include analytical measurements that focus on texture (e.g., Farinograph, TA. TXPlus, or the like). The analytical testing methods may include methods to measure the rheological and viscosity properties of the materials using a rotational shear or extensional rheometer and viscometer, respectively. Different viscometers may be used for measurement of fluid viscosity including, but not limited to, rotational, vibrational, falling sphere, or U-tube viscometers.

The formula profile may be associated with a formulation of the individual ingredients that make up the product. The formula profile may be received from a formula database that contains a full or partial formulation of a confectionery product or component including ingredients and percentages thereof.

In step 320, molecular descriptors may be determined from the molecular profile, and may include properties and/or categories such as, composition, electronic, geometric, topological, and quantum mechanical properties, among others. The molecular descriptors may include 2D descriptors, 3D descriptors, or combinations thereof. In some cases, the molecular descriptors may be received from a molecule database comprised of single small molecule data or macromolecule data along with calculated chemical predictors from the chemistry development kit (CDK). In some examples, a molecular descriptor may be based on a combination of molecular descriptors and may be classified as a hybrid descriptor. For example, a hybrid descriptor may be computed based on a combination of molecular descriptors from one or more of the categories described above.

In step 330, once the molecular descriptors are determined or received, material descriptors for the product may be determined based on the molecular descriptors for the ingredient profile. For example, a material descriptor may be determined for each individual ingredient contained in the ingredient profile.

In step 340, once the material descriptors are determined, formula descriptors for the product may be determined based on the material descriptors and the formula profile. In examples, the formula descriptors are input into a machine-learning model to train the machine-learning model to measure various functional properties of the product.

In step 350, a target set of functional properties for the product may be generated using a machine learning model based on the formula descriptors.

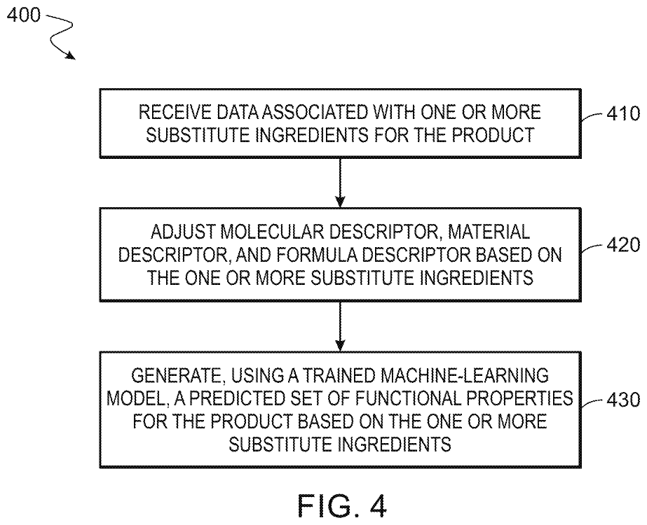

Once the machine-learning model is trained, one or more substitute ingredients for the product may be received to predict functional properties through the trained machine-learning model based on the one or more substitute ingredients. This step is explained in more detail with respect to FIG. 4.

FIG. 4 depicts a flowchart of an exemplary method 400 for using a trained machine-learning model to predict a functional property according to various embodiments. The method 400 may be performed by the property prediction platform 12 of FIG. 1.

In step 410, data associated with one or more substitute ingredients may be received. The substitute ingredients may include one or more substitute ingredients for one or more replacement ingredients of an ingredient profile of a product. The substitute ingredients may include one or more of a different weight composition of one or more like ingredients contained in the ingredient profile or one or more new ingredients not contained in the ingredient profile. Based on the substitute ingredients received, a formula profile of the product may be adjusted.

In step 420, molecular descriptors, material descriptors, and formula descriptors of the product may be adjusted based on the substitute ingredients.

In step 430, using the trained machine-learning model, a predicted set of functional properties for the product may be generated based on the one or more substitute ingredients. The predicted set of functional properties may be compared against the measured functional properties of the product. Depending on the similarity of the predicted set of functional properties and the measured set of functional properties, a substitute ingredient profile including the substitute ingredients may be generated for the product.

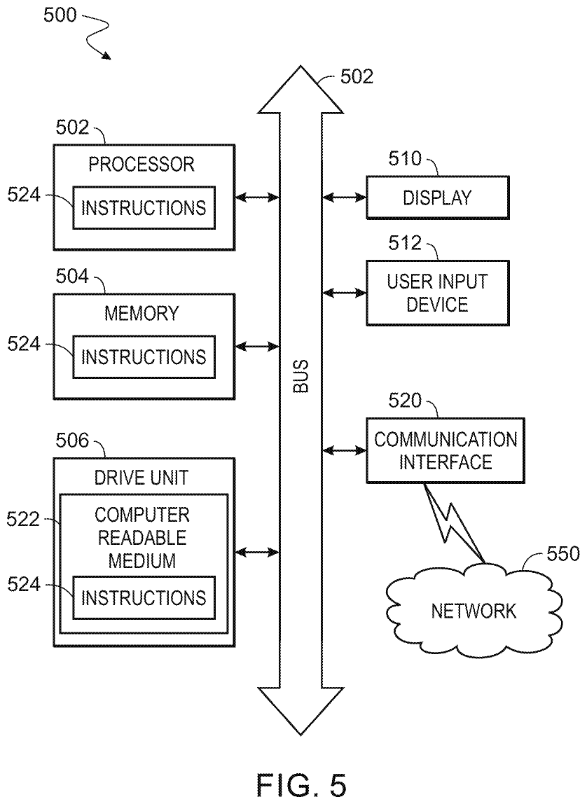

FIG. 5 illustrates an implementation of a computer system 500 that may execute techniques presented herein according to various embodiments. The computer system 500 can include a set of instructions that can be executed to cause the computer system 500 to perform any one or more of the methods or computer based functions disclosed herein. The computer system 500 may operate as a standalone device or may be connected, e.g., using a network, to other computer systems or peripheral devices.

Unless specifically stated otherwise, as apparent from the following discussions, it is appreciated that throughout the specification, discussions utilizing terms such as “processing,” “computing,” “calculating,” “determining”, analyzing” or the like, refer to the action and/or processes of a computer or computing system, or similar electronic computing device, that manipulate and/or transform data represented as physical, such as electronic, quantities into other data similarly represented as physical quantities.

In a similar manner, the term “processor” may refer to any device or portion of a device that processes electronic data, e.g., from registers and/or memory to transform that electronic data into other electronic data that, e.g., may be stored in registers and/or memory. A “computer,” a “computing machine,” a “computing platform,” a “computing device,” or a “server” may include one or more processors.

In a networked deployment, the computer system 500 may operate in the capacity of a server or as a client user computer in a server-client user network environment, or as a peer computer system in a peer-to-peer (or distributed) network environment. The computer system 500 can also be implemented as or incorporated into various devices, such as a personal computer (PC), a tablet PC, a set-top box (STB), a personal digital assistant (PDA), a mobile device, a palmtop computer, a laptop computer, a desktop computer, a communications device, a wireless telephone, a land-line telephone, a control system, a camera, a scanner, a facsimile machine, a printer, a pager, a personal trusted device, a web appliance, a network router, switch or bridge, or any other machine capable of executing a set of instructions (sequential or otherwise) that specify actions to be taken by that machine. In a particular implementation, the computer system 500 can be implemented using electronic devices that provide voice, video, or data communication. Further, while a single computer system 500 is illustrated, the term “system” shall also be taken to include any collection of systems or sub-systems that individually or jointly execute a set, or multiple sets, of instructions to perform one or more computer functions.

As illustrated in FIG. 5, the computer system 500 may include a processor 502, e.g., a central processing unit (CPU), a graphics processing unit (GPU), or both. The processor 502 may be a component in a variety of systems. For example, the processor 502 may be part of a standard personal computer or a workstation. The processor 502 may be one or more general processors, digital signal processors, application specific integrated circuits, field programmable gate arrays, servers, networks, digital circuits, analog circuits, combinations thereof, or other now known or later developed devices for analyzing and processing data. The processor 502 may implement a software program, such as code generated manually (i.e., programmed).

The computer system 500 may include a memory 504 that can communicate via a bus 508. The memory 504 may be a main memory, a static memory, or a dynamic memory. The memory 504 may include, but is not limited to computer readable storage media such as various types of volatile and non-volatile storage media, including but not limited to random access memory, read-only memory, programmable read-only memory, electrically programmable read-only memory, electrically erasable read-only memory, flash memory, magnetic tape or disk, optical media and the like. In one implementation, the memory 504 includes a cache or random-access memory for the processor 502. In alternative implementations, the memory 504 is separate from the processor 502, such as a cache memory of a processor, the system memory, or other memory. The memory 504 may be an external storage device or database for storing data. Examples include a hard drive, compact disc (“CD”), digital video disc (“DVD”), memory card, memory stick, floppy disc, universal serial bus (“USB”) memory device, or any other device operative to store data. The memory 504 is operable to store instructions executable by the processor 502. The functions, acts or tasks illustrated in the figures or described herein may be performed by the programmed processor 502 executing the instructions stored in the memory 504. The functions, acts or tasks are independent of the particular type of instructions set, storage media, processor or processing strategy and may be performed by software, hardware, integrated circuits, firm-ware, micro-code and the like, operating alone or in combination.

Likewise, processing strategies may include multiprocessing, multitasking, parallel processing and the like.

As shown, the computer system 500 may further include a display unit 510, such as a liquid crystal display (LCD), an organic light emitting diode (OLED), a flat panel display, a solid-state display, a cathode ray tube (CRT), a projector, a printer or other now known or later developed display device for outputting determined information. The display 510 may act as an interface for the user to see the functioning of the processor 502, or specifically as an interface with the software stored in the memory 504 or in the drive unit 506.

Additionally or alternatively, the computer system 500 may include an input device 512 configured to allow a user to interact with any of the components of system 500. The input device 512 may be a number pad, a keyboard, or a cursor control device, such as a mouse, or a joystick, touch screen display, remote control, or any other device operative to interact with the computer system 500.

The computer system 500 may also or alternatively include a disk or optical drive unit 506. The disk drive unit 506 may include a computer-readable medium 522 in which one or more sets of instructions 524, e.g., software, can be embedded. Further, the instructions 524 may embody one or more of the methods or logic as described herein. The instructions 524 may reside completely or partially within the memory 504 and/or within the processor 502 during execution by the computer system 500. The memory 504 and the processor 502 also may include computer-readable media as discussed above.

In some systems, a computer-readable medium 522 includes instructions 524 or receives and executes instructions 524 responsive to a propagated signal so that a device connected to a network 550 can communicate voice, video, audio, images, or any other data over the network 550. Further, the instructions 524 may be transmitted or received over the network 550 via a communication port or interface 520, and/or using a bus 508. The communication port or interface 520 may be a part of the processor 502 or may be a separate component. The communication port 520 may be created in software or may be a physical connection in hardware. The communication port 520 may be configured to connect with a network 550, external media, the display 510, or any other components in system 500, or combinations thereof. The connection with the network 550 may be a physical connection, such as a wired Ethernet connection or may be established wirelessly as discussed below. Likewise, the additional connections with other components of the system 500 may be physical connections or may be established wirelessly. The network 550 may alternatively be directly connected to the bus 508.

While the computer-readable medium 522 is shown to be a single medium, the term “computer-readable medium” may include a single medium or multiple media, such as a centralized or distributed database, and/or associated caches and servers that store one or more sets of instructions. The term “computer-readable medium” may also include any medium that is capable of storing, encoding, or carrying a set of instructions for execution by a processor or that cause a computer system to perform any one or more of the methods or operations disclosed herein. The computer-readable medium 522 may be non-transitory, and may be tangible.

The computer-readable medium 522 can include a solid-state memory such as a memory card or other package that houses one or more non-volatile read-only memories. The computer-readable medium 522 can be a random-access memory or other volatile re-writable memory. Additionally or alternatively, the computer-readable medium 522 can include a magneto-optical or optical medium, such as a disk or tapes or other storage device to capture carrier wave signals such as a signal communicated over a transmission medium. A digital file attachment to an e-mail or other self-contained information archive or set of archives may be considered a distribution medium that is a tangible storage medium. Accordingly, the disclosure is considered to include any one or more of a computer-readable medium or a distribution medium and other equivalents and successor media, in which data or instructions may be stored.

In an alternative implementation, dedicated hardware implementations, such as application specific integrated circuits, programmable logic arrays and other hardware devices, can be constructed to implement one or more of the methods described herein. Applications that may include the apparatus and systems of various implementations can broadly include a variety of electronic and computer systems. One or more implementations described herein may implement functions using two or more specific interconnected hardware modules or devices with related control and data signals that can be communicated between and through the modules, or as portions of an application-specific integrated circuit. Accordingly, the present system encompasses software, firmware, and hardware implementations.

The computer system 400 may be connected to one or more networks 550. The network 550 may define one or more networks including wired or wireless networks. The wireless network may be a cellular telephone network, an 802.11, 802.16, 802.20, or WiMax network. Further, such networks may include a public network, such as the Internet, a private network, such as an intranet, or combinations thereof, and may utilize a variety of networking protocols now available or later developed including, but not limited to TCP/IP based networking protocols. The network 550 may include wide area networks (WAN), such as the Internet, local area networks (LAN), campus area networks, metropolitan area networks, a direct connection such as through a Universal Serial Bus (USB) port, or any other networks that may allow for data communication. The network 550 may be configured to couple one computing device to another computing device to enable communication of data between the devices. The network 550 may generally be enabled to employ any form of machine-readable media for communicating information from one device to another. The network 550 may include communication methods by which information may travel between computing devices. The network 550 may be divided into sub-networks. The sub-networks may allow access to all of the other components connected thereto or the sub-networks may restrict access between the components. The network 550 may be regarded as a public or private network connection and may include, for example, a virtual private network or an encryption or other security mechanism employed over the public Internet, or the like.

In accordance with various implementations of the present disclosure, the methods described herein may be implemented by software programs executable by a computer system. Further, in an exemplary, non-limited implementation, implementations can include distributed processing, component/object distributed processing, and parallel processing. Alternatively, virtual computer system processing can be constructed to implement one or more of the methods or functionality as described herein.

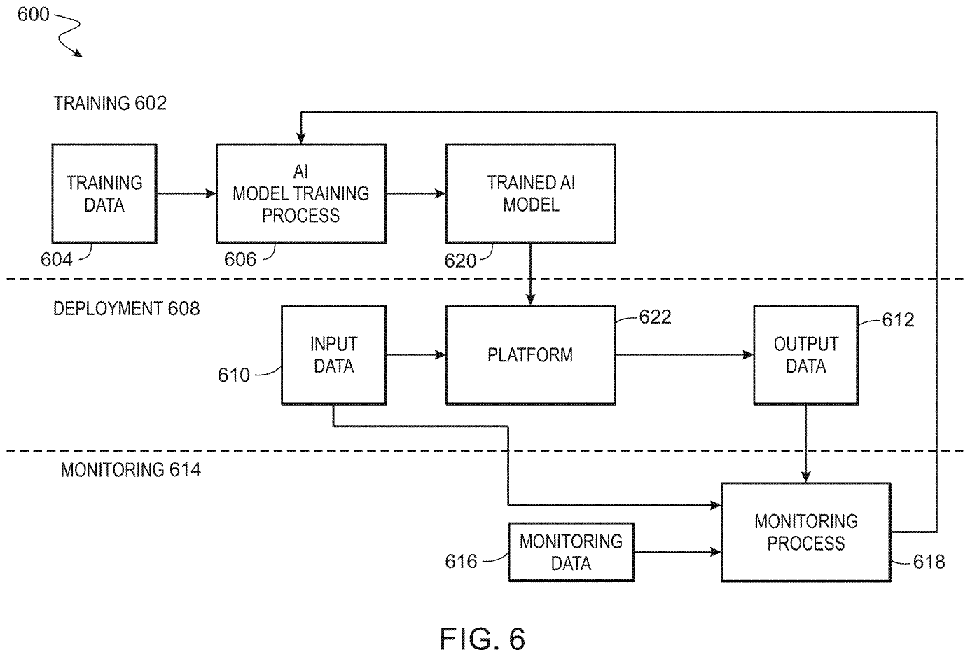

FIG. 6 is a diagram of training, deploying, and updating an artificial intelligence (AI) model. The platform 622 may generate, store, train, and/or use the AI model 620. According to an embodiment, the platform 622 may include the AI model 620 and/or instructions associated with the AI model 620. For example, the platform 622 may include instructions for generating the AI model 620, training the AI model 620, using the AI model 620, etc. According to an embodiment, a system or device other than the platform 622 may be used to generate and/or train the AI model 620. For example, a system or device may include instructions for generating the AI model 620, and/or instructions for training the AI model 620. The system or device may provide a resulting trained AI model 620 to the platform 622 for use.

As shown in FIG. 6, according to an embodiment, the process 1100 may include a training phase 602, a deployment phase 608, and a monitoring phase 614. In the training phase 602, at operation 606, the process 1000 may include receiving and processing training data 604 to generate a trained AI model 620. The training data 604 may be generated, received, or otherwise obtained from internal and/or external resources.

Generally, the AI model 620 may include a set of variables (e.g., nodes, neurons, filters, or the like) that are tuned (e.g., weighted, biased, or the like) to different values via the application of the training data 604. According to an embodiment, the training process at operation 606 may employ supervised, unsupervised, semi-supervised, and/or reinforcement learning processes to train the AI model 620. According to an embodiment, a portion of the training data 604 may be withheld during training and/or used to validate the trained AI model 620.

For supervised learning processes, the training data 604 may include labels or scores that may facilitate the training process by providing a ground truth. For example, the labels or scores may indicate an output of the AI model 620. Training may proceed by feeding a training dataset including the training data 604 into the AI model 620. The AI model 620 may have variables set at initialized values (e.g., at random, based on Gaussian noise, based on pre-trained values, or the like). The AI model 620 may generate an output based on the training dataset being input to the AI model 620. The output may be compared with the corresponding label or score (e.g., the ground truth) indicating the known output, which may then be back-propagated through the AI model 620 to adjust the values of the variables. This process may be repeated for a plurality of samples at least until a determined loss or error is below a predefined threshold. According to an embodiment, some of the training data 604 may be withheld and used to further validate or test the trained AI model 620.

For unsupervised learning processes, the training data 604 may not include pre-assigned labels or scores to aid the learning process. Instead, unsupervised learning processes may include clustering, classification, or the like, to identify naturally occurring patterns in the training data 604. As an example, the training data may be clustered into groups based on identified similarities and/or patterns. K-means clustering or K-Nearest Neighbors may also be used, which may be supervised or unsupervised. Combinations of K-Nearest Neighbors and an unsupervised cluster technique may also be used. For semi-supervised learning, a combination of training data 604 with pre-assigned labels or scores and training data 604 without pre-assigned labels or scores may be used to train the AI model 620.

When reinforcement learning is employed, an agent (e.g., an algorithm) may be trained to make a decision from the training data 604 through trial and error. For example, based on making a decision, the agent may then receive feedback (e.g., a positive reward if the prediction was above a predetermined threshold), adjust its next decision to maximize the reward, and repeat until a loss function is optimized.

After being trained, the trained AI model 620 may be stored and subsequently applied by the platform 622 during the deployment phase 608. For example, during the deployment phase 608, the trained AI model 620 executed by the platform 622 may receive input data 610. During the deployment phase 608, the trained AI model 620 may perform one or more operations as described herein.

After being deployed, the trained AI model 620 may be monitored during the monitoring phase 614. For example, during the monitoring phase 614, the AI model 620 may generate monitoring data 616 that is used to monitor the trained AI model 620. The monitoring data 616 may include data that identifies an output as determined by an operator. During process 618, the monitoring data 616 may be analyzed along with the predicted output data 612 and input data 610 to determine an accuracy of the trained AI model 620. According to an embodiment, based on the analysis, the process 1100 may return to the training phase 602, where at operation 606 values of one or more variables of the model may be adjusted to improve the accuracy of the AI model 620.

The example process 1100 described above is provided merely as an example, and may include additional, fewer, different, or differently arranged aspects than depicted in FIG. 6.

FIG. 6 describes the training, deployment, and monitoring associated with a trained AI model 620. According to an embodiment, one or more other trained AI model 620s may be applied. Each of the trained AI model 620s may include similar training, deployment, and/or monitoring phases as described above for the trained AI model 620 in FIG. 6, however the particular types of training data, input data, output data, and monitoring data may be different.

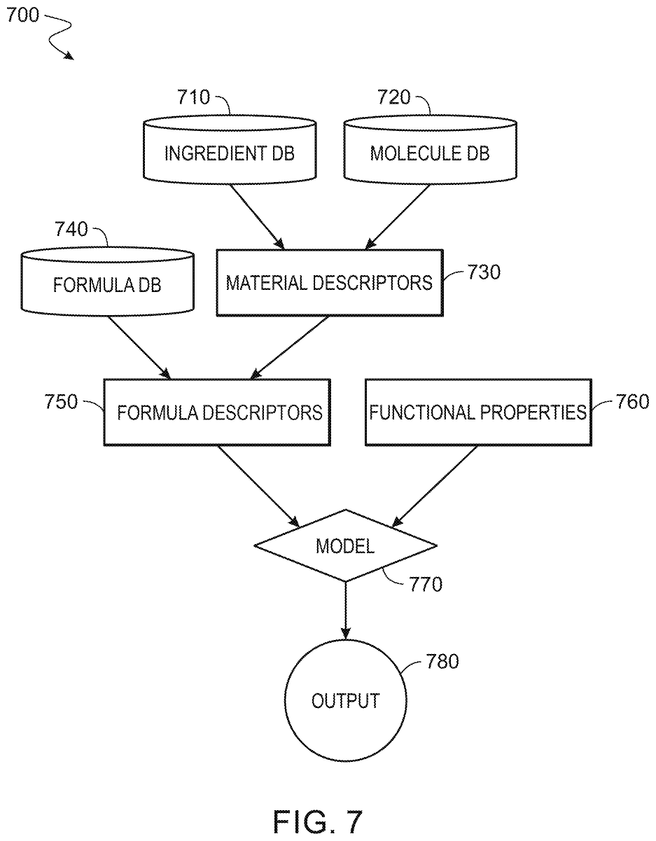

FIG. 7 is a diagram 700 of an example process for training the AI model 120. As shown in FIG. 7 by reference number 710, the platform 620 may receive information from an ingredient database. The Ingredient Database may store information identifying ingredients used in the manufacture of confectionery products and the compositions of the ingredients. The compositions may include carbohydrate profile, fat profile, protein composition, or the like. As shown by reference number 720, the platform 620 may receive information from a molecule database. The molecule database may store information identifying single small molecule or macromolecules along with calculated chemical predictors from the Chemistry Development Kit (CDK). The molecular descriptors include composition properties, electronic properties, geometric properties, topological properties, quantum mechanical properties, or the like. As shown by reference number 730, the platform 620 may generate material descriptors based on the information received from the ingredient database and the information received from the molecule database. As shown by reference number 740, the platform 622 may receive information from a formula database. As shown by reference number 750, the platform 622 may generate formula descriptors based on the information received from the formula database and the material descriptors. As shown by reference number 760, the platform 622 may receive functional properties. As shown by reference number 770, the platform 622 may input the formula descriptors and the functional properties into the AI model 620, and train the AI model 620. As shown by reference number 780, the AI model 620 may be configured to generate an output.

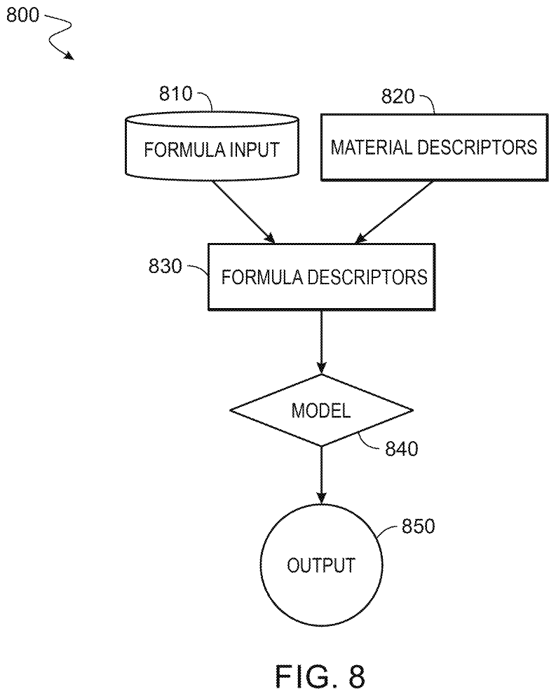

FIG. 8 is a diagram 800 of an example process for new formula performance prediction. As shown in FIG. 8 by reference number 810, the platform 622 may receive an input formula. As shown by reference number 820, the platform 622 may receive material descriptors. As shown by reference number 830, the platform 622 may generate a formula descriptor based on the input formula and the material descriptors. As shown by reference number 840, the platform 622 may input the formula descriptor into the AI model 620. As shown by reference number 850, the AI model 620 may generate an output.

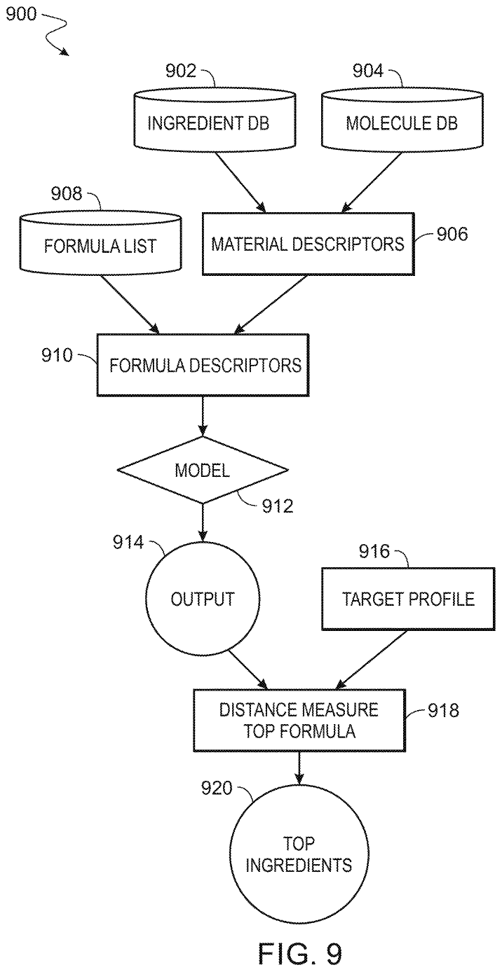

FIG. 9 is a diagram 900 of an example process for generating information identifying a new ingredient for a product. As shown in FIG. 9 by reference number 902, the platform 622 may receive information from the ingredient database. As shown by reference number 904, the platform 622 may receive information from the molecule database. As shown by reference number 906, the platform 622 may generate material descriptors based on the information received from the ingredient database and the molecule database. As shown by reference number 908, the platform 622 may receive information from a formula list. As shown by reference number 910, the platform 622 may generate formula descriptors. As shown by reference number 912, the platform 622 may input the formula descriptors into the AI model 620. As shown by reference number 914, the AI model 620 may generate an output. As shown by reference number 916, the platform 620 may receive a target profile. As shown by reference number 918, the platform 622 may evaluate a desired performance profile using a distance metric (e.g., Euclidean distance, cosine distance, or the like). As shown by reference number 920, the platform 622 may determine a list of top ingredients for the new product.

Although the present specification describes components and functions that may be implemented in particular implementations with reference to particular standards and protocols, the disclosure is not limited to such standards and protocols. For example, standards for Internet and other packet switched network transmission (e.g., TCP/IP, UDP/IP, HTML, HTTP) represent examples of the state of the art. Such standards are periodically superseded by faster or more efficient equivalents having essentially the same functions. Accordingly, replacement standards and protocols having the same or similar functions as those disclosed herein are considered equivalents thereof.

It will be understood that the steps of methods discussed are performed in one embodiment by an appropriate processor (or processors) of a processing (i.e., computer) system executing instructions (computer-readable code) stored in storage. It will also be understood that the disclosed embodiments are not limited to any particular implementation or programming technique and that the disclosed embodiments may be implemented using any appropriate techniques for implementing the functionality described herein. The disclosed embodiments are not limited to any particular programming language or operating system.

It should be appreciated that in the above description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the Detailed Description are hereby expressly incorporated into this Detailed Description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those skilled in the art. For example, in the following claims, any of the claimed embodiments can be used in any combination.

Thus, while certain embodiments have been described, those skilled in the art will recognize that other and further modifications may be made thereto without departing from the spirit of the invention, and it is intended to claim all such changes and modifications as falling within the scope of the invention. For example, functionality may be added or deleted from the block diagrams and operations may be interchanged among functional blocks. Steps may be added or deleted to methods described within the scope of the present invention.

The above disclosed subject matter is to be considered illustrative, and not restrictive, and the appended claims are intended to cover all such modifications, enhancements, and other implementations, which fall within the true spirit and scope of the present disclosure. Thus, to the maximum extent allowed by law, the scope of the present disclosure is to be determined by the broadest permissible interpretation of the following claims and their equivalents, and shall not be restricted or limited by the foregoing detailed description. While various implementations of the disclosure have been described, it will be apparent to those of ordinary skill in the art that many more implementations are possible within the scope of the disclosure. Accordingly, the disclosure is not to be restricted except in light of the attached claims and their equivalents.

What is claimed is:

1. A computer-implemented method for predicting a property of a product with substitute ingredients, the method comprising:
receiving, by one or more processors, data associated with a product, the data including an ingredient profile of the product, a molecular profile associated with the ingredient profile, and a formula profile for the product based on the ingredient profile;
determining, by the one or more processors, a set of molecular descriptors for the product based on the molecular profile;
determining, by the one or more processors, a set of material descriptors for the product based on the set of molecular descriptors;
determining, by the one or more processors, a set of formula descriptors for the product based on the set of material descriptors and the formula profile; and
generating, by the one or more processors, a measured set of functional properties for the product through training of a machine-learning model based on the one or more formula descriptors;
receiving, by the one or more processors, one or more substitute ingredients for the product to be evaluated with the machine-learning model; and
generating, by the one or more processors, a predicted set of functional properties for the product through use of the machine-learning model based on the one or more substitute ingredients.

2. The computer-implemented method of claim 1, further comprising:
prior to generating the predicted set of functional properties and after receiving the one or more substitute ingredients, adjusting, by the one or more processors, the formula profile and the set of molecular descriptors based on the one or more substitute ingredients;
adjusting, by the one or more processors, the set of material descriptors based on the adjusted set of molecular descriptors; and
adjusting, by the one or more processors, the set of formula descriptors based on the adjusted set of material descriptors and the adjusted formula profile,
wherein generating the predicted set of functional properties through use of the machine-learning model is further based on the adjusted set of formula descriptors.

3. The computer-implemented method of claim 1, further comprising:
generating a substitute ingredient profile including the one or more substitute ingredients for the product based on a similarity between the predicted set of functional properties and the measured set of functional properties,
wherein the similarity is determined based on an evaluation of a distance metric between the predicted set of functional properties and the measured set of functional properties, the distance metric including one or more of: a euclidean distance metric or a cosine distance metric.

4. The computer-implemented method of claim 1, wherein the one or more substitute ingredients include one or more of: a different weight composition of one or more like ingredients contained in the ingredient profile or one or more new ingredients not contained in the ingredient profile.

5. The computer-implemented method of claim 1, wherein the product includes one of: a toffee candy, a chocolate, a nougat, or a caramel.

6. The computer-implemented method of claim 1, wherein the machine-learning model includes a quantitative structure-activity relationship (QSAR) model, the QSAR model including one of: a linear regression model, a stepwise linear regression model, a partial least squares regression model, a principal components regression model, a support vector regression model, neural networks, a multivariate adaptive regression model, a multivariate linear regression model, or a random forest model.

7. The computer-implemented method of claim 1, wherein the machine-learning model includes one of: a polynomial regression model, a generalized additive model, a Bayesian additive regression tree model (BART), a classification and regression tree model (CART), a multi-layer perceptron model (MLP), a recurrent neural network model (RNN), or a convolutional neural network model (CNN).

8. The computer-implemented method of claim 1, wherein training the machine-learning model includes use of a train/test split, use of hyperparameters tuned using a multi-fold repeated cross validation, or use of a variable selection technique.

9. The computer-implemented method of claim 8, wherein the multi-fold repeated cross validation ranges in value between 5 and 10 repeated cross validations, and wherein the variable selection technique includes one or more of: a recursive feature elimination, a backward or forward stepwise selection, simulated annealing, or a genetic algorithm search.

10. The computer-implemented method of claim 1, wherein the ingredient profile includes ingredient data of individual ingredients used in manufacturing the product, the ingredient data including composition of the individual ingredients, the composition including one or more of: a carbohydrate profile, a fat profile, or a protein composition.

11. The computer-implemented method of claim 1, wherein the set of molecular descriptors includes molecular data associated with individual ingredients contained in the ingredient profile, the molecular data including one or more of:
composition properties of the individual ingredients, electronic properties of the individual ingredients, geometric properties of the individual ingredients, topological properties of the individual ingredients, or quantum mechanical properties of the individual ingredients.

12. The computer-implemented method of claim 1, wherein the formula profile includes partial or full formulation data of the product based on the ingredient profile, the partial or full formulation data including a percentage composition of individual ingredients contained in the ingredient profile.

13. The computer-implemented method of claim 1, wherein the measured set of functional properties and the predicted set of functional properties include organoleptic properties of the product.

14. A system for predicting a property of a product with substitute ingredients, the system comprising:
a memory configured to store instructions; and
one or more processors configured to execute the instructions to perform operations comprising:
receiving data associated with a product, the data including an ingredient profile of the product, a molecular profile associated with the ingredient profile, and a formula profile for the product based on the ingredient profile;
determining a set of molecular descriptors for the product based on the molecular profile;
determining a set of material descriptors for the product based on the set of molecular descriptors;
determining a set of formula descriptors for the product based on the set of material descriptors and the formula profile; and
generating a measured set of functional properties for the product through training of a machine-learning model based on the one or more formula descriptors;
receiving one or more substitute ingredients for the product to be evaluated with the machine-learning model; and
generating a predicted set of functional properties for the product through use of the machine-learning model based on the one or more substitute ingredients.

15. The system of claim 14, wherein the operations further comprise:
prior to generating the predicted set of functional properties and after receiving the one or more substitute ingredients,
adjusting the formula profile and the set of molecular descriptors based on the one or more substitute ingredients;
adjusting the set of material descriptors based on the adjusted set of molecular descriptors; and
adjusting the set of formula descriptors based on the adjusted set of material descriptors and the adjusted formula profile,
wherein generating the predicted set of functional properties through use of the machine-learning model is further based on the adjusted set of formula descriptors.

16. The system of claim 14, wherein the operations further comprise:
generating a substitute ingredient profile including the one or more substitute ingredients for the product based on a similarity between the predicted set of functional properties and the measured set of functional properties,
wherein the similarity is determined based on an evaluation of a distance metric between the predicted set of functional properties and the measured set of functional properties, the distance metric including one or more of: a euclidean distance metric or a cosine distance metric

17. The system of claim 14, wherein the one or more substitute ingredients include one or more of: a different weight composition of one or more like ingredients contained in the ingredient profile or one or more new ingredients not contained in the ingredient profile.

18. The system of claim 14, wherein the product includes one of: a toffee candy, a chocolate, a nougat, or a caramel.

19. The system of claim 14, wherein the machine-learning model includes a quantitative structure-activity relationship (QSAR) model, the QSAR model including one of: a linear regression model, a stepwise linear regression model, a partial least squares regression model, a principal components regression model, a support vector regression model, neural networks, a multivariate adaptive regression model, a multivariate linear regression model, or a random forest model.

20. A non-transitory computer-readable medium storing instructions that, when executed by one or more processors of a system for predicting a property of a product with substitute ingredients, cause the system to perform operations comprising:
receiving data associated with a product, the data including an ingredient profile of the product, a molecular profile associated with the ingredient profile, and a formula profile for the product based on the ingredient profile;
determining a set of molecular descriptors for the product based on the molecular profile;
determining a set of material descriptors for the product based on the set of molecular descriptors;
determining a set of formula descriptors for the product based on the set of material descriptors and the formula profile; and
generating a measured set of functional properties for the product through training of a machine-learning model based on the one or more formula descriptors;
receiving one or more substitute ingredients for the product to be evaluated with the machine-learning model; and
generating a predicted set of functional properties for the product through use of the machine-learning model based on the one or more substitute ingredients.
